(19) United States
(12) Patent Application Publication
(10) Pub. No.: US 2025/0288190 A1
(43) Pub. Date: Sep. 18, 2025

(54) MEDICAL DEVICE HANDLES, COMPONENTS, AND RELATED METHODS OF MANUFACTURING AND/OR ASSEMBLING

(71) Applicant: Boston Scientific Scimed, Inc., Maple Grove, MN (US)

(72) Inventors: Todd ROBIDA, Sherborn, MA (US); Thomas BIANCHI, Palmer, MA (US)

(73) Assignee: Boston Scientific Scimed, Inc., Maple Grove, MN (US)

(21) Appl. No.: 19/075,025

(22) Filed: Mar. 10, 2025

Related U.S. Application Data
(60) Provisional application No. 63/565,608, filed on Mar. 15, 2024.

(51) Int. Cl. A61B 1/005; A61B 1/015; A61B 1/018; A61M 25/01

(52) U.S. Cl. CPC A61B 1/0052; A61B 1/015; A61B 1/018; A61M 25/0136

(57) ABSTRACT

A medical device may comprise a handle including a port and a shaft extending from a portion of the handle. A first portion the shaft may include a plurality of first lumens and a second lumen. The plurality of first lumens and the second lumen may be defined by a single, integrally formed body. A second portion of the shaft defining the second lumen may extend proximally of proximalmost openings of each of the plurality of first lumens. A proximal end of the second portion of the shaft may be coupled to the port.

### CROSS-REFERENCE TO RELATED APPLICATIONS

This application claims the benefit of priority to U.S. Provisional Application No. 63/565,608, filed on Mar. 15, 2024, which is incorporated by reference herein in its entirety.

### TECHNICAL FIELD

This disclosure generally relates to medical device handles, components, and related methods. In some aspects, this disclosure relates to adapters and/or internal components of a handle that couple a working channel of a shaft to a port (e.g., a biopsy port) on an exterior of the handle, and related methods of manufacturing and/or assembling.

### BACKGROUND

Medical devices are often inserted into the body to perform a therapeutic and/or diagnostic procedure inside a subject's body. An example of such a device is an endoscope or other type of scope or insertion device, which includes a handle and an insertion portion extending distally from the handle. The insertion portion is introduced into the body for diagnostic or therapeutic purposes. For example, an insertion portion of an endoscope is inserted into the subject's body through an opening (e.g., a natural opening or an incision) and is delivered to a site inside the body, for example, through a body lumen. In one example, an endoscope may be inserted into a subject's mouth and advanced through the subject's esophagus.

The insertion portion of an endoscope or other insertion device includes a shaft that extends from outside the body to the treatment site. The shaft includes a working channel or internal lumen. The shaft often also includes a number of other channels or lumens, for example, for delivering irrigation, for applying suction, for receiving pull or deflection wires, electrical connections, or other components, etc. The shaft is coupled to a handle, which may control various aspects of the shaft, including movement of the pull or deflection wires, the delivery of irrigation, the application of suction, the activation/deactivation of the electrical connections, etc. The working channel or internal lumen of the shaft is often coupled (e.g., fluidly coupled) to a port on the handle, such as a biopsy port on an exterior of the handle.

### SUMMARY

Examples of this disclosure relate to, among other things, medical devices having a handle and a shaft or insertion portion. For example, examples of this disclosure relate to devices, components, and methods of coupling a portion of the insertion portion or shaft (e.g., a working channel or lumen) to a portion of the handle (e.g., to a biopsy port).

According to one aspect, a medical device may comprise a handle including a port and a shaft extending from a portion of the handle. A first portion the shaft may include a plurality of first lumens and a second lumen. The plurality of first lumens and the second lumen may be defined by a single, integrally formed body. A second portion of the shaft defining the second lumen may extend proximally of proximalmost openings of each of the plurality of first lumens. A proximal end of the second portion of the shaft may be coupled to the port.

Any of the aspects disclosed herein may include any of the following features, alone or in any combination or subcombination. The second lumen may be a working channel. The plurality of first lumens may include at least one fluid delivery lumen. The plurality of first lumens may further include a plurality of wire lumens. The second portion of the shaft may have a smaller width than the first portion of the shaft. The second lumen may be an only lumen of the second portion of the shaft. A coupling tube may be received within a lumen of the port and within the second lumen. A distal portion of the coupling tube may include an enlarged portion or a groove. The shaft may include a wing portion. The wing portion may include a first, attached distal end and a second, free end. A hub may at least partially surround the shaft. The hub may be coupled to the port by a beam. The beam may be a first beam. A second beam may extend distally from the port. The second portion of the shaft may include a recess. The first beam or the second beam may be received within the recess. The second portion of the shaft may be keyed to the port. A third portion of the shaft, proximal of the second portion of the shaft, may include a third lumen.

In another aspect, a medical device may comprise a handle including a port and a shaft extending from a portion of the handle. The shaft may include a proximal portion and a distal portion. The distal portion may include a plurality of lumens. The proximal portion may include only a single lumen of the plurality of lumens. The shaft may be single, integrally formed body. The proximal portion may be coupled to the port.

Any of the aspects disclosed herein may have any of the following features, alone or in any combination or subcombination. A coupling tube may be received within a lumen of the port and within the single lumen of the proximal portion. A distal portion of the coupling tube may include an enlarged portion or a groove. A hub may at least partially surround the shaft. The hub may be coupled to the port by a beam.

In another aspect, a medical assembly may comprise: a frame including a port, a hub, and a beam extending from the port to the hub; and a shaft. The shaft may include a proximal portion and a distal portion. The shaft may be a single, integrally formed body defining a plurality of lumens. The proximal portion may have a smaller width than the distal portion. The proximal portion may be coupled to the port. The hub may be coupled to the proximal portion or the distal portion.

Any of the aspects disclosed herein may have any of the following features, alone or in any combination. The proximal portion may have a single lumen of the plurality of lumens. A remainder of the plurality of lumens may have proximal ends that are distal to a proximal end of the single lumen.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1A is a perspective view of a proximal portion of a medical device, according to aspects of this disclosure.

FIG. 1B is a perspective view of a distal portion of the medical device, according to aspects of this disclosure.

FIG. 2A illustrates an interior portion of a handle of the medical device, and FIG. 2B is a cross-sectional view of a portion of an insertion portion or shaft of the medical device, according to aspects of this disclosure.

FIG. 3 illustrates a proximal portion of the insertion portion or shaft of the medical device, according to aspects of this disclosure.

FIGS. 4A and 4B illustrate perspective and cross-sectional views, respectively, of a portion of the handle of the medical device, according to aspects of this disclosure.

FIGS. 5A-5F illustrate perspective views of various coupling elements that may be used in the portion of the handle of the medical device, according to aspects of this disclosure.

FIGS. 6A-8 depict aspects of various exemplary shafts and shaft assemblies including hubs and/or ports, according to aspects of this disclosure.

FIGS. 9A-10 depict aspects of frame assemblies for supporting various shafts in the handle of the medical device, according to aspects of this disclosure.

FIGS. 11-13 depict aspects of various exemplary shafts, according to aspects of this disclosure.

FIGS. 14A and 14B depict an exemplary frame assembly for supporting a shaft in the handle, according to aspects of this disclosure.

FIGS. 15 and 16 depict aspects of two exemplary shafts, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when introducing the device into the subject. Proximal and distal directions are labeled with arrows marked “P” and “D,” respectively, throughout various figures.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic. Additionally, terms that indicate the geometric shape of a component/surface encompass both exact and approximate shapes.

Although a target site is discussed herein as being in the subject's gastrointestinal tract, this disclosure is not so limited, as the target site may be any internal lumen, organ, cavity, or other tissue within the subject. Additionally, although endoscopes are referenced herein, it will be appreciated that the disclosure encompasses various devices that may be inserted into a body of a subject, such as ureteroscopes, cholangioscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

This disclosure is described with reference to exemplary medical devices, components, assemblies and/or systems having a handle and an insertion portion. It should be noted that reference to any particular device and/or any particular procedure is provided only for convenience and not intended to limit the disclosure. A person of ordinary skill in the art would recognize that the concepts underlying the disclosed devices and application methods may be utilized in any suitable procedure, medical or otherwise. The assemblies and systems described herein may be used in conjunction with other types of medical devices. This disclosure may be understood with reference to the following description and the appended drawings, wherein like elements are referred to with the same reference numerals.

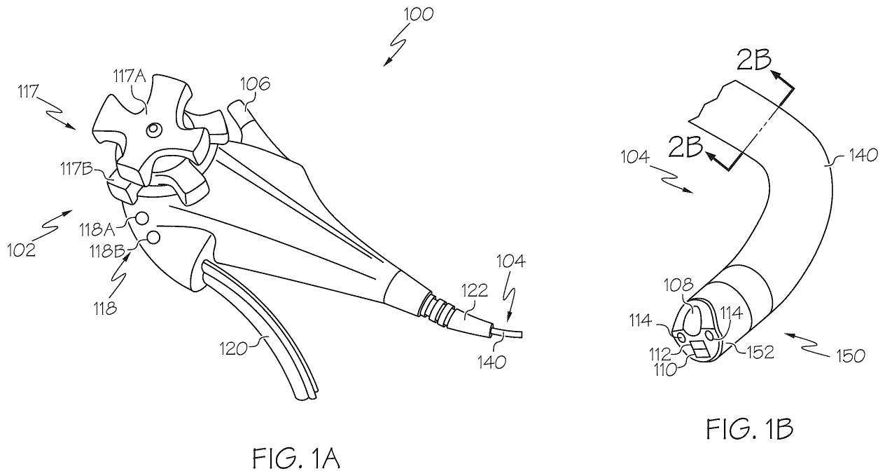

Referring to FIG. 1A, a medical device 100 according to an exemplary embodiment is shown. Medical device 100 includes a handle 102 and an insertion portion 104 (including, e.g., a shaft 140). Insertion portion 104 may be connected to and extend from a distal portion of handle 102. Insertion portion 104 may terminate distally in a distal end 150, as shown in FIG. 1B. In embodiments, the distal end of medical device 100 may include an end cap 152 that incorporates components at the distal end. Additionally, handle 102 includes an opening or a port 106 (e.g., a biopsy port), which may be fluidly connected to a working channel 108 and/or one or more lumens of insertion portion 130. As discussed in detail below, a portion of insertion portion 104 may be formed of shaft 140 (e.g., a multi-lumen extrusion), and only a portion of the shaft may extend proximally to port 106. For example, a first portion of shaft 140 may include a plurality of lumens, and a second portion of shaft may extend proximally to port 106 and may include only a subset of the lumens (e.g., one lumen).

Additionally, the distal end of medical device 100 (e.g., end cap 152) may include one or more illumination devices 110 (e.g., lights, LEDs, optical fibers, etc.) and/or one or more visualization devices 112 (e.g., cameras, imagers, etc.). Insertion portion 104 (e.g., shaft 140 and end cap 152) may also include one or more channels or lumens, for example, one or more fluid delivery lumens 114. Furthermore, as discussed in detail below, insertion portion 104 may include one or more additional channels or lumens, for example, to receive one or more pull or deflection wires, optical fibers, cables, electrical wires, communication wires, etc.

Handle 102 may include one or more actuators 117, for example, a first actuator 117A and a second actuator 117B. Actuators 117A, 117B may be manipulated (e.g., rotated about their respective axis) to control the deflection, movement, position, etc. of one or more portions of insertion portion 104, for example, distal end 150. In these aspects and as discussed in detail below, actuators 117A, 117B may be connected to one or more pull or deflection wires or cables that extend through one or more portions of insertion portion 104 and connect to distal end 150 of insertion portion 104.

Rotation of first actuator 117A and/or second actuator 117B may cause a portion of insertion portion 104 (e.g., distal end 150) to bend, for example, via an articulating joint (not shown). For example, rotation of first actuator 117A in a first direction (e.g., clockwise) may pull a first pull wire proximally, such that distal end 150 deflects in a first deflection direction (e.g., up). Rotation of first actuator 117A in a second direction (e.g., counterclockwise) may pull a second pull wire proximally, such that distal end 150 deflects in a second deflection direction (e.g., down). The second deflection direction may be opposite to the first deflection direction. Additionally, in some examples, rotation of second actuator 117B a first direction (e.g., clockwise) may pull a third pull wire proximally, such that distal end 150 deflects in a third deflection direction (e.g., left). Rotation of second actuator 117B in a second direction (e.g., counterclockwise) may pull a fourth pull wire proximally, such that distal end 150 deflects in a fourth deflection direction (e.g., right).

Although not shown, handle 102 may include one or more locking mechanisms to secure one or more of first actuator 117A and/or second actuator 117B, and thus help to secure the position of distal end 150. Although actuators 117A, 117B are illustrated as knobs, actuators 117A, 117B may instead be buttons, levers, sliders, etc. Additionally or alternatively, handle 102 may include one or more additional actuators (e.g., buttons, knobs, levers, etc.) to, for example, close or open an end effector, rotate an end effector about a longitudinal axis, raise or lower an elevator to move a device delivered through the working channel, capture an image and/or video, and/or provide other functionality to an end effector and/or distal end 150.

Handle 102 may also include one or more valves 118, for example, a first valve 118A and a second valve 118B. Although two valves (i.e., first valve 118A and second valve 118B) are shown, handle 102 may include additional (e.g., a third valve, a fourth valve, etc.) or fewer valves (e.g., no valves or only one of first valve 118A or second valve 118B). In some embodiments, first valve 118A may be configured to control the supply of air and/or water through insertion portion 104 to distal end 150. Second valve 118B may be configured to control the application of suction through insertion portion 104 to distal end 150. Additional valves may be used, for example, to control the application of one or more medicines, agents, materials, etc. from distal end 150.

An umbilicus 120 may extend from handle 102 (e.g., from a proximal portion of handle 102). Umbilicus 120 may be removably coupled (e.g., directly or indirectly) to a processing unit (not shown). The processing unit may be configured to process information (e.g., sensor data, imaging data, light data, etc.) received from medical device 100. In some aspects, the processing unit may be a controller associated with medical device 100. Illumination device(s) 110 and/or imaging device(s) 112 may be electrically coupled (e.g., directly or indirectly) to the processing unit, for example, via one or more wires and/or cables extending through insertion portion 104, through handle 102, and through umbilicus 120. For example, umbilicus 120 may include one or more electrical cables and/or light cables for coupling to the processing unit via, e.g., a removable connector.

Although not shown, the processing unit may include or be coupled to a visual output (e.g., an embedded monitor or screen, or an external or separate monitor or screen). Although not shown, umbilicus 120 may additionally or alternatively include one or more lumens for supplying a gas or a liquid, or applying suction or negative pressure, to handle 102 and/or insertion portion 104. For example, the gas or the liquid may be applied, or delivered, through one or more channels, or lumens, of medical device 100.

Handle 102 may be coupled to a proximal portion of insertion portion 104 (e.g., shaft 140) via a strain relief portion 122. Strain relief portion 122 may extend from a distal end of handle 102, and may overlap with a proximal portion of shaft 140, for example, helping to maintain the coupling of handle 102 to insertion portion 104.

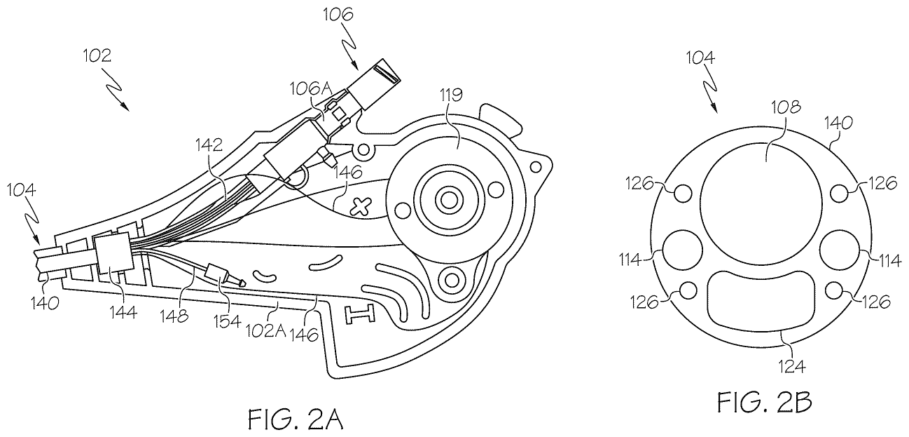

FIG. 2A is a side view of internal components of handle 102 and a portion of insertion portion 104, without umbilicus 120, and FIG. 2B is a lateral cross-sectional view of a portion of insertion portion 104. As shown in FIG. 2A, handle 102 includes a handle body 102A that encloses one or more components of medical device 100. For example, handle body 102A may be formed of two halves that are coupled together (e.g., adhered, welded, snap fit, etc.) to form handle body 102A. Moreover, handle 102 includes port 106, for example, including a port body 106A. A portion of shaft 140 may extend proximally to be positioned within a portion of handle body 102A. Furthermore, a portion of shaft 140 (e.g., a skived portion 142 or a portion that forms working channel 108, shown in FIG. 2B) extends through handle body 102A to be coupled to a portion of port 106 (e.g., to port body 106A). Skived portion 142 may include only a single complete lumen—working channel 108. In other words, working channel 108 may be an only complete lumen of skived portion 142.

Skived portion 142 may be separated from the remainder of shaft 140, for example, via cutting with a razor blade, trimming tool, etc. The separation of skived portion 142 from the remainder of shaft 140 may be done, for example, under microscope or magnification. In these aspects, the additional lumens or channels of shaft 140 may have exposed proximal ends to receive or be coupled to other components of medical device 100. Skived portion 142 may have a smaller perimeter than more distal portions of shaft 140.

Additionally, a portion of shaft 140 may be coupled to or extend through an adapter or hub 144. Hub 144 may help to securely couple shaft 140 to handle 102. In some aspects and as discussed in detail below, cut portions of shaft 140 may be retained, for example, to help couple shaft 140 to hub 144.

As shown, medical device 100 includes one or more articulation, deflection, or pull wires 146, which may be coupled to one or more of actuators 117 (FIG. 1A), for example, to an internal actuator portion 119. Rotation of one or more of actuators 117 may also rotate internal actuator portion 119. As discussed above, rotation or manipulation of actuators 117 may thus also control one or more of pull wires 146 to control an articulation, movement, or position of a portion of insertion portion 104 (e.g., distal end 150). Although only one internal actuator portion 119 and two pull wires are shown, it is noted that handle 102 may include two internal actuator portions (e.g., each coupled to one of actuators 117A and 117B), and that medical device 100 may include four pull wires 146 (e.g., with two pull wires 146 coupled to each internal actuator portion 119). Additionally, each pull wire 146 may extend through a lumen or channel within shaft 140 (as discussed below), for example, through respective proximal openings (e.g., adjacent to hub 144 and where skived portion 142 extends from the remainder of shaft 140).

Medical device 100 also includes one or more fluid tubes, for example, coupling portions of shaft 140 to one or more portions of handle 102. For example, medical device 100 may include a fluid tube 148, such as an irrigation tube, which may convey a fluid such as water or air to distal end 150. In some aspects, fluid tube 148 may be coupled (e.g., at a proximal ends) to an irrigation or air hub or adapter 154. Although not shown, medical device 100 may also include one or more additional fluid tubes for irrigation (water), air (insufflation) and/or or suction (aspiration). The one or more fluid tubes 148 may extend through or couple one or more fluid lumens in shaft 140 to one or more valves 118 (e.g., one tube extending to first valve 118A, and one tube extending to second valve 118B). In these aspects, valve(s) 118 may control the delivery of fluid and/or application of suction to a treatment site via one or more channels or lumens in shaft 140. In some aspects, adapter 154 may help to couple valve(s) 118 (FIG. 1A) to fluid tube 148 or other tubes.

Although not shown, medical device 100 may include one or more communication wires or cables. The one or more communication wires or cables may extend from a portion of shaft 140 through one or more portions of handle 102. For example, the one or more communication wires or cables may extend from shaft 140 to (or through) umbilicus 120 (FIG. 1A). The one or more communication wires or cables may extend through one or more channels or lumens in shaft 140, for example, to couple one or more components of distal end 150 (e.g., illumination device(s) 110 and/or visualization device(s) 112) to handle 102 and/or umbilicus 120.

As shown in FIG. 2B, insertion portion 104 includes shaft 140. As mentioned, shaft 140 may be a multi-lumen extrusion, for example, formed of polytetrafluoroethylene (PTFE) and/or additive-filled polymers, which may help to lubricate to one or more lumens (e.g., working channel 108). Shaft 140 may be formed of a single piece of material, e.g., to form a single, integral body. Lumens of shaft 140 may be formed integrally from shaft 140. For example, shaft 140 may be a multi-lumen extrudate.

Shaft 140 includes working channel 108. Working channel 108 may be positioned in a central portion of shaft 140, or may be slightly off-center (as shown). Shaft 140 also may include one or more fluid delivery lumens 114. A communication lumen 124 may receive one or more cables, wires, etc. to electrically connect one or more of illumination device(s) 110 and/or imaging device(s) 112 to handle 102 and/or umbilicus 120. Shaft 140 also includes one or more wire lumens 126, for example, to movably receive respective pull wires 146 (FIG. 2A). Wire lumens 126 may be evenly or unevenly spaced around outer portions of shaft 140, for example, above or below one or more of lumens 114 and/or adjacent to (e.g., radially outside of) one or more of working channel 108 and/or communication lumen 124.

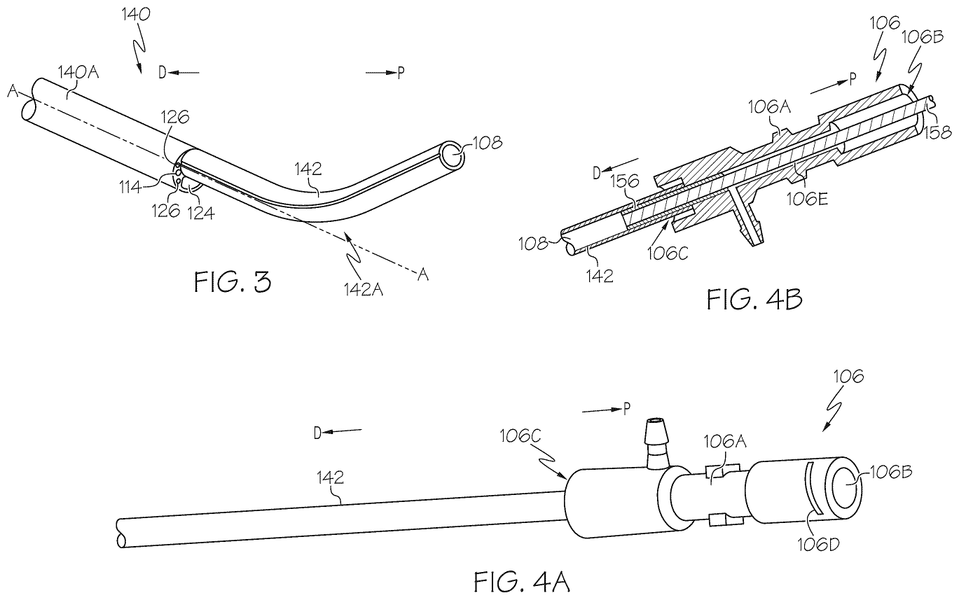

FIG. 3 illustrates a proximal portion of shaft 140 separated from other components or portions of medical device 100. As shown, skived portion 142 extends proximally of the remainder of shaft 140 (e.g., a shaft body 140A), and forms a proximal portion of working channel 108. As discussed above, skived portion 142 may be formed by skiving (or otherwise cutting or removing) portions of shaft 140 other than a portion defining working channel 108. For example, skived portion 142 may be formed by removing lumens 114, communication lumen 124, and one or more wire lumens 126 from a proximal portion of shaft 140 (e.g., shaft body 140A).

A proximal end of skived portion 142 may be coupled to port 106 (FIGS. 1A and 2A), such that working channel 108 is fluidly and sealingly coupled to port 106. Furthermore, in some aspects, skived portion 142 may include one or more bent or angled portions 142A, such that a proximal end of skived portion 142 is positioned above or otherwise offset from a longitudinal axis A of shaft 140. In these aspects, skived portion 142 and a proximal portion of working channel 108 are isolated from the remaining portion (e.g., shaft body 140A) and the channels or lumens (lumens 114, communication lumen 124, and one or more wire lumens 126).

In aspects, skived portion 142 helps to provide an instrument or accessory channel with a consistent shape, size, material, etc., for example, with a smooth internal surface to receive and guide one or more instruments or accessories from port 106, through working channel 108, and to distal end 150. Furthermore, the proximal ends or openings of the remainder of the channels or lumens are exposed or open at their proximalmost ends, for example, at the proximal end of shaft body 140A, as shown in FIG. 3. In these aspects, the proximalmost ends of lumens 114, communication lumen 124, and wire lumen(s) 126 may be distal to a proximalmost end of working channel 108.

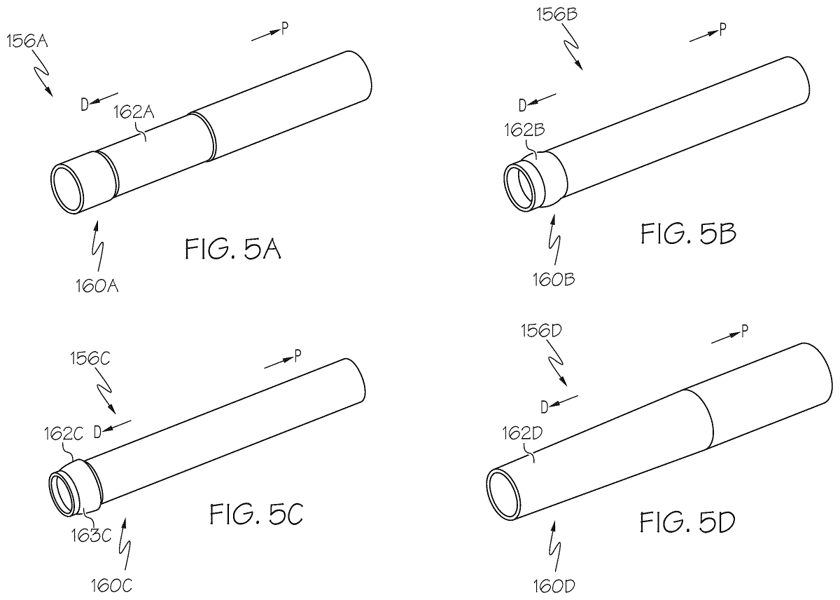

FIGS. 4A and 4B illustrate various aspects of skived portion 142 being coupled to port 106. As shown in FIG. 4A, port 106 may include a port body 106A, which includes a proximal opening 106B and a distal opening 106C. An instrument (e.g., an accessory device with an end effector) may be delivered through port body 106A (e.g., through proximal opening 106B) and into working channel 108 of skived portion 142, such that the instrument is also delivered through a length of skived portion 142 and through a length of shaft 140 to extend out of the distal opening of working channel 108. A proximal portion of port body 106A may include one or more surface features 106D (FIG. 4A), for example, threading or grooves, which may help a user couple a proximal portion of the instrument to port body 106A.

As shown in FIG. 4B, skived portion 142 maybe coupled to port 106 (e.g., port body 106A) via a coupling tube 156. Coupling tube 156 may be generally cylindrical, or alternatively may be one or more other shapes or surface features, as discussed below. Coupling tube 156 may be a hypotube, for example, approximately 10-20 gauge hypotube, approximately 12-16 gauge hypotube, approximately 14 or approximately 15 gauge hypotube. A proximal portion of coupling tube 156 may be positioned within a portion of port body 106A, for example, via distal opening 106C. Port body 106A includes a port lumen 106E, for example, fluidly connecting proximal opening 106B to distal opening 106C. In some aspects, port lumen 106E and coupling tube 156 may be sized and/or shaped such that at least a portion of coupling tube 156 may be received within port lumen 106E, for example, to form a press fit coupling. Alternatively and as discussed below, coupling tube 156 may be securely coupling to port 106 (e.g., in port lumen 106E) with one or more adhesives. In alternatives, coupling tube 156 may be overmolded or otherwise formed with port body 106A. Furthermore, as discussed below, skived portion 142 may be coupled to port body 106A via one or more adhesives, for example, via injecting the one or more adhesives around the components. In some aspects, distal opening 106C may be larger than coupling tube 156 and the proximal end of skived portion 142 in order to form a volume (i.e., a space) to receive (e.g., contain) the one or more adhesives to help couple the components.

Additionally, a proximal portion of skived portion 142 may be coupled to a distal portion of coupling tube 156. For example, a proximal portion of skived portion 142 may be positioned on an outer surface (e.g., a radially outer surface) of coupling tube 156. As discussed below, the distal portion of coupling tube 156 may include one or more surface features to help secure skived portion 142 to coupling tube 156, and thus to help secure skived portion 142 to port 106. Furthermore, one or more adhesives may be applied, delivered, or otherwise positioned around one or more of port body 106A, coupling tube 156, and/or skived portion 142 to help secure skived portion 142 to coupling tube 156, and thus to help secure skived portion 142 to port body 106A. In some aspects, a proximal portion of skived portion 142 may include one or more surface features to help the one or more adhesives couple skived portion 142 to port body 106A. For example, if shaft 140 (and thus skived portion 142) is formed of PTFE, the proximal portion of skived portion 142 of shaft 140 may be etched (e.g., chemically etched) to help the one or more adhesives couple skived portion 142 to port body 106A.

Additionally, as mentioned above, skived portion 142 and/or shaft 140 may be coupled to hub 144 (see FIG. 2A), and the portion of skived portion 142 and/or shaft 140 that is coupled to hub 144 may also be etched or otherwise treated to help couple skived portion 142 and/or shaft 140 to hub 144. For example, one or more portions of shaft 140 and/or skived portion 142 may be treated by one or more of: laser texturing, film wrapping, swaging (e.g., swaging a marker band over the proximal end, with the marker band being formed of a material with increased bonding or coupling properties), inserting a barbed marker band into the proximal end of skived portion 142 (e.g., with the barbed marker band being formed of a material with increased bonding or coupling properties), wire wrapping, including barbs in one or more channels (e.g., in communication lumen 124), including one or more anchors in one or more channels (e.g., in communication lumen 124), including one or more stops (e.g., in port 106 and/or hub 144), wrapping portions of shaft 140 (e.g., portions of shaft 140 that do not form skived portion 142) to help anchor portions of shaft 140 to hub 144, including one or more wings, “ears,” or other extensions on portions of shaft 140, etc.

Additionally, during the coupling of the various components, port 106 may receive a mandrel 158, as shown in FIG. 4B. Mandrel 158 may help to secure or otherwise hold or maintain an alignment of port body 106A, skived portion 142, and coupling tube 156 during assembly. In some examples, port body 106A may include a recessed portion for receiving coupling tube 156, such that an inner diameter of a lumen of port body 106A is a same as a lumen of coupling tube 156. Mandrel 158 may be sized and shaped to be received within a lumen of coupling tube 156. For example, mandrel 158 may help to maintain the positioning of the various components during the delivery and/or curing of an adhesive, may help to prevent buckling or other changes in shape of skived portion 142 during heating (e.g., heat shrinking, heated drying or curing of the adhesive, etc.), or may otherwise help to retain the relative positions during assembly. Furthermore, mandrel 158 may help to prevent adhesive(s) from flowing into the inner lumen (e.g., working channel 108) of skived portion 142, coupling tube 156B, and/or port lumen 106E.

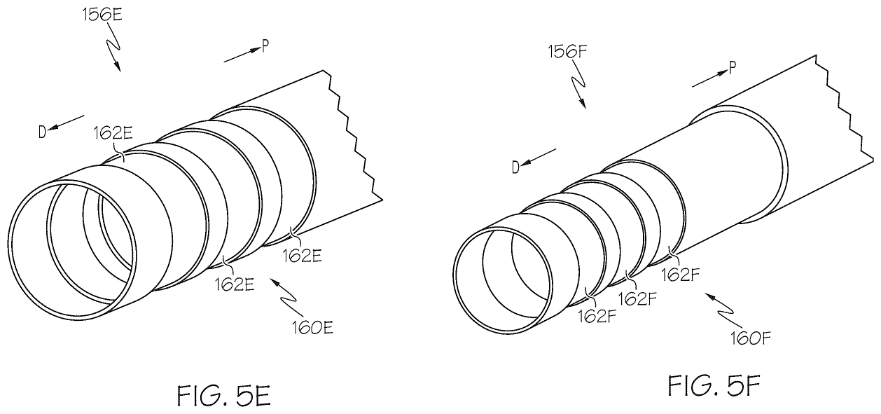

FIGS. 5A-5F illustrate various exemplary coupling tubes that may be used to help couple skived portion 142 to port body 106A, as discussed above. For example, FIG. 5A illustrates a coupling tube 156A. A distal portion 160A of coupling tube 156A may include one or more swaged, indented, or reduced diameter (e.g., radially inward) portions 162A. In some aspects, reduced diameter portion(s) 162A may help to allow glue or other adhesive(s) to be positioned between coupling tube 156A and skived portion 142 (e.g., an inner diameter portion of skived portion 142). In some aspects, vacuum pressure may be used (e.g., applied to proximal opening 106B of port 106) to help draw the adhesive into or along reduced diameter portion(s) 162A to help secure coupling tube 156A to port 106. Furthermore, in some aspects, reduced diameter portion(s) 162A may help to receive a portion of skived portion 142, for example, when a proximal portion of skived portion 142 is heat shrunk or otherwise formed onto coupling tube 156A.

FIG. 5B illustrates another coupling tube 156B. A distal portion 160B of coupling tube 156B may include one or more beaded or enlarged portions 162B. In some aspects, beaded portion(s) 162B may help to form a seal, for example, between coupling tube 156B and the proximal portion of skived portion 142. Additionally, beaded portion(s) 162B may help to form the seal with a low or reduced insertion or coupling force, for example, to position the proximal portion of skived portion 142 over or around distal portion 160B of coupling tube 156B.

FIG. 5C illustrates another coupling tube 156C. A distal portion 160C of coupling tube 156C may include one or more tapered beaded or enlarged portions 162C. As shown in FIG. 5C, a distal portion 163C of tapered beaded portion(s) 162C may be tapered or angled radially inward, for example, forming a flat surface. The remainder of beaded portion(s) 162C (e.g., the proximal portion) may be curved or rounded. In some aspects, the tapering of distal portion 163C may help to couple skived portion 142 on, over, or around distal portion 160C. In some aspects, tapered beaded portion(s) 162C may help to form a seal, for example, between coupling tube 156C and the proximal portion of skived portion 142. Tapered beaded portion(s) 162C may help to form the seal with a low or reduced insertion or coupling force, for example, to position the proximal portion of skived portion 142 on, over, or around distal portion 160C of coupling tube 156C.

FIG. 5D illustrates another exemplary coupling tube 156D. A distal portion 160D of coupling tube 156D may include an angled or tapered (e.g., radially inward) portion 162D. In some aspects, coupling tube 156D (or any of the other coupling tubes disclosed herein) may be a hypotube having an outer diameter of approximately 0.06-0.09 inches, or approximately 0.076 inches. An inner diameter of coupling tube 156D (or any of the other coupling tubes disclosed herein) may be approximately 0.06 inches to approximately 0.08 inches, or approximately 0.068 inches to approximately 0.072 inches. In some examples, a distalmost end of coupling tube 156D may have an inner diameter of at least 0.068 inches. The tapering (or reduction in diameter) along tapered portion 162D may be consistent (as shown). Alternatively, although not shown, the tapering may be inconsistent, for example, with a more gradual taper along a proximal portion of tapered portion 162D and a steeper (e.g., less gradual) taper along a distal portion of tapered portion 162D. In some aspects, tapered portion 162D may help to allow glue or other adhesive(s) to be positioned between coupling tube 156D and skived portion 142 (e.g., an inner diameter portion of skived portion 142). In some aspects, vacuum pressure may be used (e.g., applied to proximal opening 106B of port 106) to help draw the adhesive into or along tapered portion 162D and secure coupling tube 156D to port 106. Tapered portion 162D may help to couple skived portion 142 on, over, or around distal portion 160D.

FIG. 5E illustrates a coupling tube 156E. A distal portion 160E of coupling tube 156E may include a plurality of swaged, indented, or reduced diameter (e.g., radially inward) grooves 162E. In these aspects, coupling tube 156E may be a 15 gauge hypotube, for example, with an inner diameter of approximately 0.072 inches to 0.069 inches and a wall thickness of approximately 0.0015 inches. Grooves 162E may be approximately 0.002 inches deep, for example, being indented into the internal lumen of coupling tube 156E. In some aspects, reduced diameter grooves 162E may help to allow glue or other adhesive(s) to be positioned between coupling tube 156E and skived portion 142 (e.g., an inner diameter portion of skived portion 142). In some aspects, vacuum pressure may be used (e.g., applied to proximal opening 106B of port 106) to help draw the adhesive into or along reduced diameter grooves 162E to help to secure coupling tube 156E to port 106. Furthermore, in some aspects, reduced diameter grooves 162E may help to receive a portion of skived portion 142, for example, when a proximal portion of skived portion 142 is heat shrunk or otherwise formed onto coupling tube 156E.

FIG. 5F illustrates another coupling tube 156F. A distal portion 160F of coupling tube 156F may include a plurality of swaged, indented, or reduced diameter (e.g., radially inward) grooves 162F. In these aspects, coupling tube 156F may be a 14 gauge hypotube, for example, with an inner diameter of approximately 0.083 inches to 0.069 inches and a wall thickness of approximately 0.0075 inches. Grooves 162F may be approximately 0.002 inches deep. Additionally, in some examples, grooves 162F may be formed by grinding (e.g., may be centerless grind grooves), such that grooves 162F are only on the outer surface of coupling tube 156F and do not extend radially inward on the inner surface of coupling tube 156F. For example, distal portion 160F may be formed via grinding or otherwise removing an outer portion of coupling tube 156F, with grooves 162F being formed by removing more of the outer portion of coupling tube 156F. In some aspects, reduced diameter grooves 162F may help to allow glue or other adhesive(s) to be positioned between coupling tube 156F and skived portion 142 (e.g., an inner diameter portion of skived portion 142). In some aspects, vacuum pressure may be used (e.g., applied to proximal opening 106B of port 106) to help draw the adhesive into or along reduced diameter grooves 162F. Furthermore, in some aspects, reduced diameter grooves 162F may help to receive a portion of skived portion 142, for example, when a proximal portion of skived portion 142 is heat shrunk or otherwise formed onto coupling tube 156F. In some examples, an entirety of distal portion 160F may be recessed relative to more proximal portions of coupling tube 156F.

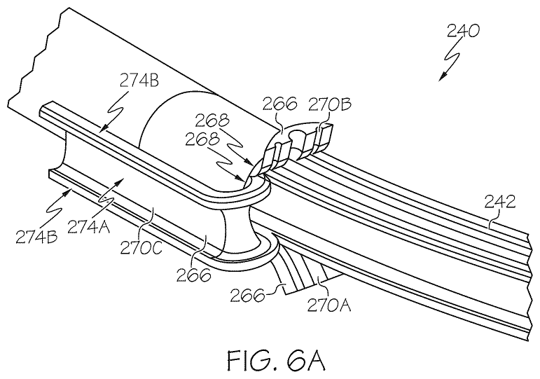

FIGS. 6A-6B illustrate various aspects of a shaft 240 and a handle 202. Shaft 240 may be coupled to one or more of a port (such as port 106, not shown) and to a hub 244. A skived portion 242 (having any of the properties of skived portion 142) may include the portion of shaft 240 that forms the working channel. Additionally, skived portion 242 may optionally include a portion of shaft 240 that forms one or more additional lumens (e.g., communication lumen, fluid lumen(s), wire lumen(s)).

Portions of shaft 240 that have been cut or skived away from skived portion 242 may form a winged portion having one or more wings 266 (i.e., wing portions). First, attached (i.e., fixed) ends 268 of wings 266 may remain coupled to (not cut away from) shaft 240. Radially inner portions of wings 266 may be cut or skived away from skived portion 242. Wings 266 may form strips or flaps that may be movable (e.g., bendable or foldable) with respect to skived portion 242. Although three wings 266 are shown, it will be appreciated that any suitable number of wings 266 may be utilized. Wings 266 may have any suitable width around a circumference/perimeter of shaft 240.

Wings 266 may include a first wing 270A, a second wing 270B, and a third wing 270C. Although three wings 266 are shown, any shaft 240 may include any suitable number of wings 266. First wing 270A and second wing 270B may have a smaller longitudinal length than third wing 270C. The cuts used to form wings 270A and 270B may have a same or similar shape. For example, wings 270A and 270B may have a ridged/grooved radially inner surface. An outer surface of skived portion 242 from which wings 270A and 270B were cut may also have a ridged/grooved outer surface. The ridged/groove surfaces may be formed by the cuts that form wings 270A and 270B extending through fluid and/or steering lumens. In contrast, third wing 270B may include a smooth, recessed central portion 274A and outer ridges 274B. Recessed portion 274A may correspond to a surface of an electronics lumen, for example. Electronic elements 271 (e.g., wires or cables) may extend proximally from a proximal opening of the electronics lumen (described above).

Wings 270A and 270B may form “ears,” which may be folded distally and/or radially outward to help to secure shaft 240 to hub 244. In some examples, outer or inner surfaces of wings 270A, 270B may form reservoirs that may help glue to couple shaft 240 to hub 244. Second, free ends of wings 270A and 270B may be proximal of hub 244. Additionally or alternatively, any of wings 266 may help to prevent shaft 240 from being pulled out of handle 202.

Wing 270C may also be folded distally, such that a second, free end of wing 270C is within or distal to hub 244. Wing 270C may extend along an outer surface of shaft 240 that is distal to where shaft 240 has been cut or skived. A portion of shaft 240 along which wing 270C extends may have an overall non-circular shape because of a presence of wing 270C. The non-circular shape of shaft 240 and wing 270C may inhibit shaft 240 from rotating relative to hub 244. Hub 244 may extend at least partially around shaft 240 and one or more of wings 266. As shown in FIG. 6B, wing 270C may extend through a slot 273 of hub 244. Slot 273 may be sized and shaped such that wing 270C inhibits rotation of shaft 240 and, in aspects, helps to secure shaft 240 to hub 244. Slot 273 may be configured such that hub 244 only partially surrounds the shaft. In alternatives, slot 273 may be omitted, and hub 244 may define a lumen with a fully enclosed radially outer perimeter for receiving shaft 240 and wing 270C. As shown in FIG. 6B, handle body 102A may include one or more supports 103 (e.g., protrusions) for interacting with hub 244, shaft 240, and/or wing 270C to help secure hub 244 and shaft 240.

Hub 244 may be an element of a frame 280, which may include a beam 276. Beam 276 may extend toward port 106. Aspects of frames, similar to frame 280 will be discussed in further detail below. As shown in FIG. 6B, beam 276 may include a recess 276A, which may receive a support 103 of handle body 102A. Beam 276 may provide increased support and tension on skived portion 242 to increase fixation to port 106 and decrease a risk of skived portion 242 from becoming disconnected from port 106.

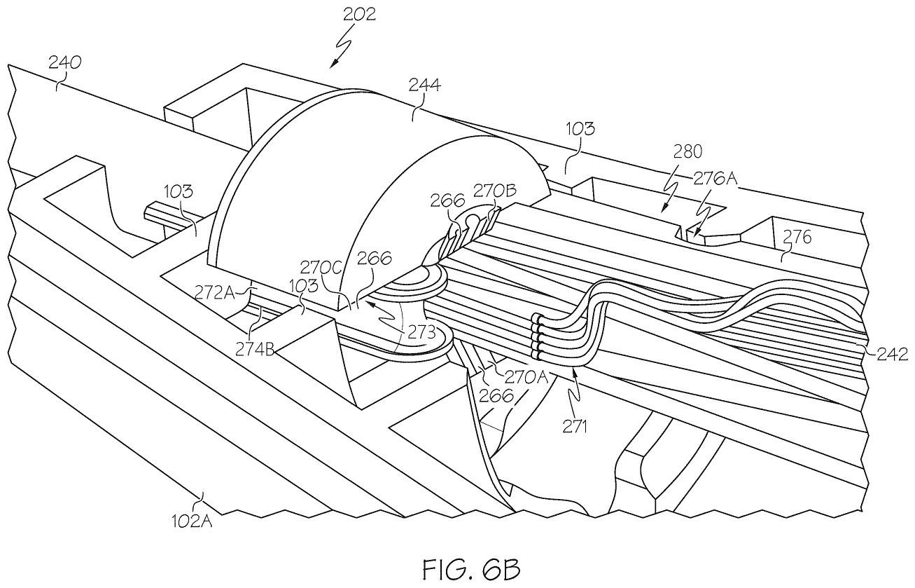

FIGS. 7A and 7B depict another shaft 340 and hub 344 of an assembly 300. Similar to shaft 240, shaft 340 may include one or more wings 366 and a skived portion 342. Skived portion 342 may be coupled to a port 306 having any of the properties of any of the ports disclosed herein. Shaft 340 may include three wings 366. Similarly to wings 266, wings 366 may be folded back to help to secure shaft 340 to a hub 344. For example, one or more of wings 366 may be folded proximally within hub 344 or one or more of wings 366 may be folded such that free end(s) of the one or more of wings 366 are proximal of hub 344.

Hub 344 may surround an entire circumference/perimeter of shaft 340. As shown in FIGS. 7A and 7B, hub 344 may include one or more recesses 344A, which may receive one or more supports 103 of handle body 102A to help secure hub 344 within handle 102.

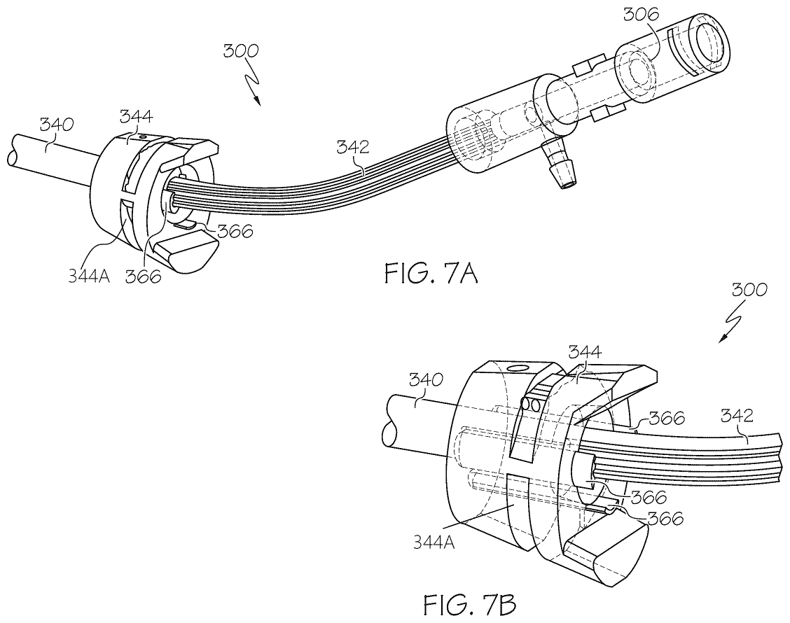

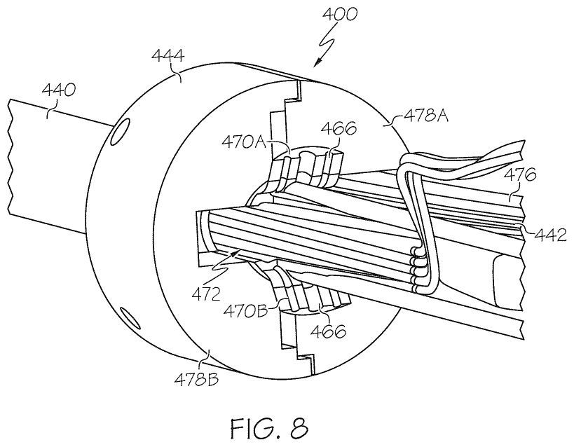

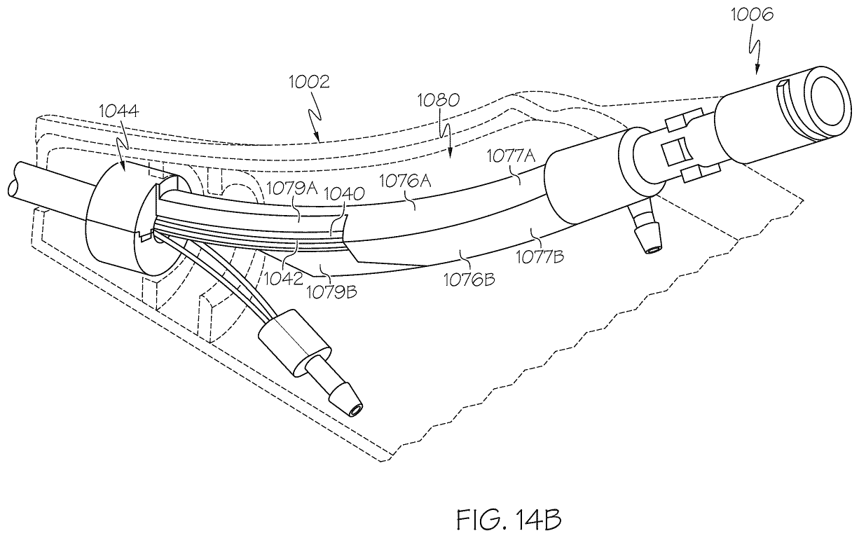

FIG. 8 shows another shaft 440 and hub 444 of an assembly 400. As discussed in further detail below with respect to FIGS. 9A and 9B, hub 444 may be an element of a frame. Similarly to shafts 240 and 340, shaft 440 may include a plurality of wings 466. For example, shaft 440 may include a first wing 470A and a second wing 470B. Wings 470A, 470B may have any of the properties of any of the wings 366 disclosed above, such as wings 270A and 270B. As shown in FIG. 8, wings 470A and 470B may have free ends that are bent distally or radially outward to help secure shaft 440 to hub 444. A skived portion 442 of shaft 440 may include a notched portion 472 (which may be similar to notch 1172 of FIG. 15, described below) that may further help to secure shaft 440 to hub 444. Further details of notched portions are discussed below with respect to FIG. 15 and notched portion 474 may include any of the features discussed with respect to FIG. 15.

Similarly to hub 344, hub 444 may surround an entire perimeter/circumference of shaft 440. Hub 444 may include a first piece 478A and a second piece 478B. Each of first piece 478A and second piece 478B may form approximately half of hub 444. First piece 478A and second piece 478B may be separate pieces that are coupled together (e.g., via friction fit, mating components, and/or adhesive) around shaft 440. As discussed in further detail below, first piece 478A may be coupled to (e.g., integrally formed with) a support beam 476 of the frame.

FIGS. 9A-9B depict a handle 502 having frame 580 that combines hub 544 with a port 506 having any of the properties of port 106. FIG. 9A shows exemplary handle 502 with a portion of a handle body 102A of exemplary handle 502 removed so that internal components of handle 502 are visible. FIG. 9B shows a cross-sectional view of handle 502, such that frame 580 is shown in cross-section. Handle 502 may include a shaft 540 having a skived portion 542. As shown in FIG. 9B, skived portion 542 may have a working channel 508 running therethrough.

Frame 580 may provide support to skived portion 542 of shaft 540. For example, frame 580 may have a curved shape so as to support a curved shape of skived portion 542 and to inhibit skived portion 542 from being subjected to tension or traction that would tend to disconnect skived portion 542 from port 506 and/or hub 544 or break seals in any such connections.

For example, frame 580 may include a first beam 576A and a second beam 576B. First beam 576B and second beam 576B may be on opposite sides of skived portion 542 (e.g., on a top and bottom of skived portion 542 in the orientation of handle 502 shown in FIGS. 9A and 9B, along a longitudinal length of skived portion 542). First beam 576A may extend distally from port 506 to hub 544 and may be coupled to both port 506 and hub 544. Second beam 576B may extend distally from port 506 toward hub 544 but may terminate proximally of hub 544. An arced segment 576C may extend between port 506 and second beam 576B. Arced segment 576C may provide further support to second beam 576B and may inhibit bending of second beam 576B relative to port 506.

A curve of skived portion 542 may be distal to where the curve would be absent frame 580. Thus, skived portion 542 may have a relatively gentler curve that may place smaller forces on skived portion 542 that would act to disconnect skived portion 542 from port 506 and/or hub 544. Furthermore, second beam 576B may counteract a loading force to help prevent buckling of skived portion 542 and/or loosening of joints between skived portion 542 and port 506 or hub 544. First beam 576A and/or second beam 576B may further protect skived portion 542 from compression forces that would tend to disconnect or loosen skived portion 542 from port 506 or hub 544.

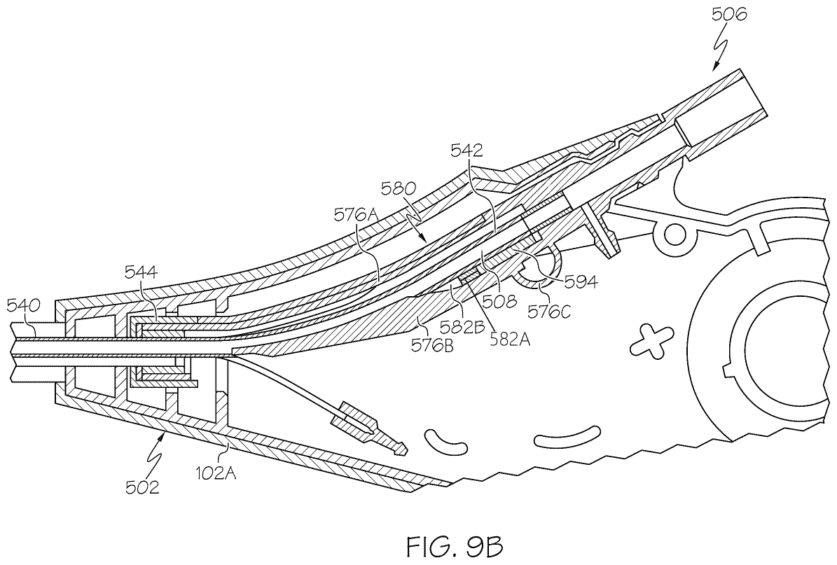

As discussed in further detail below (e.g., as described with respect to FIGS. 11-13, and in particular FIG. 11), skived portion 542 may include a shape so as to help rotationally secure skived portion 542 relative to portions of frame 580 (e.g., first beam 576A, second beam 576B, port 506, and/or hub 544). For example, skived portion 542 may include a protrusion 594 (having any of the features of a protrusion 794, discussed below with respect to FIG. 11), which may be received within port 506, proximally of a tension lock member 582A. Protrusion 594 may be integrally formed with skived portion 542 or may be a separate member. Tension lock member 582A may abut a distal surface of protrusion 594 to help to secure protrusion 594 within port 506. Tension lock member 582A may be compliant before skived portion 542 is adhered to frame 580 with an adhesive. Thus, tension lock member 582A may allow insertion of protrusion 594 into port 506 proximally of tension lock member 582A. During installation of skived portion 542 into frame 580, adhesive may be filled within a gap 582B between frame 580 and skived portion 542 to lock tension lock member 582A in place.

Furthermore, frame 580 may assist in manufacturing of handle 502. For example, frame 580 and shaft 540 (including skived portion 542) may form an assembly that may be installed into handle 502 with fewer concerns of breaking seals/connections between skived portion 542 and port 506 (and/or hub 544). Similar to hub 444, hub 544 may include two pieces and may eliminate a need for a mandrel to couple skived portion 542 to hub 544.

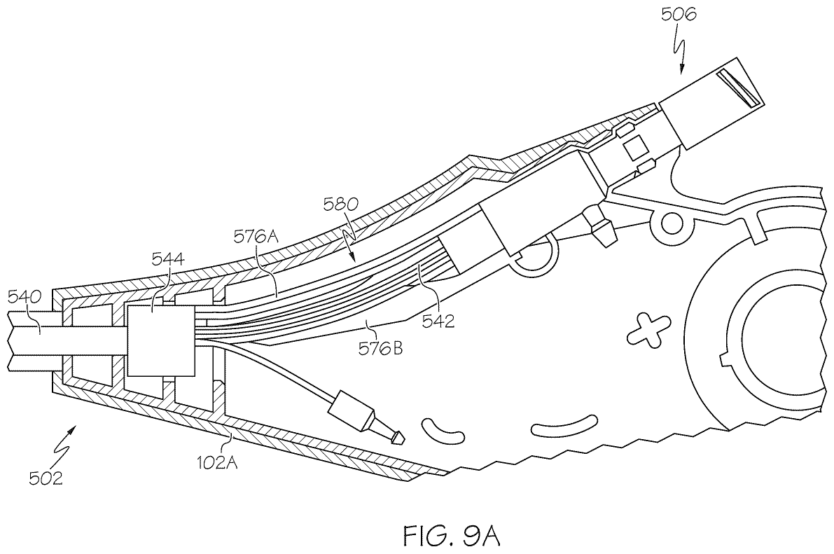

FIG. 10 shows an alternative frame 680. Frame 680 may have any of the features of frame 580 except as specified below. Frame 680 may also have any of the benefits of frame 580. Frame 680 may include a single beam 676 extending between a port 606 and a hub 644. Beam 676 may hold a desired compression of a skived portion of a shaft (including any of the skived portions discussed above or below). In other words, beam 676 may cause the skived portion to bend. A distal portion 606A of port 606, and other portions of port 606 may maintain a desired tension on the skived portion.

Frame 680 may be a single piece, formed of a single, integral piece of material. Hub 644 may include an aperture or lumen 644A for receiving a shaft having any of the properties of any of the shafts disclosed herein (either an entire portion of a shaft or a skived portion of a shaft). As shown in FIG. 10, lumen 644A may have a non-circular shape that may be keyed to a shape of the shaft. In some examples, the shaft may be skived so as to have a shape to correspond to a shape of lumen 644A. Alternatively or additionally, wings of the shaft (such as the wings discussed above) may be folded back within lumen 644A to key the shaft to lumen 644A and to inhibit rotation of the shaft relative to lumen 644A.

FIGS. 11-13 depict various skiving configurations for shafts that may help to inhibit rotation between the shafts and hubs or ports (such as any of the hubs or ports discussed above or below) and/or to prevent disconnection between the shafts and hubs or ports. Features of the shafts below may be combined in any suitable manner.

FIG. 11 depicts an exemplary shaft 740, which may be used along with aspects of the disclosure discussed above and below. As shown in FIG. 11, a distal portion 788 of shaft 740 may be tubular, having round cross-sectional shape. A proximal portion 789 of shaft 740 may include a first (distal) skived portion 742A and a second (proximal) skived portion 742B.

To form first skived portion 742A, portions 790A, 790B, and 790C of distal portion 788 may be skived away or otherwise removed. For example, portions 790A and 790B may include lumens for delivering fluid (e.g., water or air, such as fluid delivery lumens 114) and lumens for steering/articulation wires (e.g., wire lumens 126). Portion 790C may include a communication lumen for electronic elements (e.g., wires). First skived portion 742A may thus include only a portion of shaft 740 that defines a working channel 708 (e.g., walls of shaft 740 that define working channel 708).

Portions 790A and 790B may be removed from shaft 740 along an entirety of skived portion 742, including first skived portion 742A and second skived portion 742B. Portion 790C may be removed only from first skived portion 742A but may not be removed from (i.e., may be retained on) second skived portion 742B. Thus, second skived portion 742B may include a portion of shaft 740 that defines a communication lumen (having any of the properties of communication lumen 124) in distal portion 788. Second skived portion 742B may thus include two lumens-a working channel 708 and a lumen 724 that corresponds to the communication lumen in distal portion 788.

Thus, skived portion 742 may have a first side surface 792A and a second side surface 792B that extends along an entire length of skived portion 742, including first skived portion 742A and second skived portion 742B. First side surface 792A and second side surface 792B of skived portion 742 may be ridged. The ridges may be formed because lumens for delivering fluid and for containing articulation wires may be cut in half (or cut by another fraction) during skiving of first skived portion 742A and second skived portion 742B. For example, shaft 740 may be cut vertically through the lumens for delivering fluid and containing articulation wires. Thus, recesses in first side surface 792A and second side surface 792B may correspond to portions of lumens in distal portion 788 for air/water and/or articulation wires.

Skived portion 742 may also have a third side 792C that has not been skived and that extends along an entire length of skived portion 742 and may be continuous with an outer surface of distal portion 788. Third side 792C may have a curved shape that is the same as a curved shape of a corresponding surface of distal portion 788. First skived portion 742A may also include a fourth outer surface 792D. Fourth outer surface 792D may correspond to an outer surface of a wall 793 between working channel 708 and the communication and/or lumen 724. Fourth outer surface 792D may have any suitable shape, including a smooth, curved shape.

Second skived portion 742B may have a protrusion 794 that includes lumen 724 and protrudes radially outward relative to fourth outer surface 792D. An outer surface of protrusion 794 may correspond to an outer surface of distal portion 788 that is adjacent to the communication lumen and/or lumen 724 and may be curved. For example, an outer surface of protrusion 794 may be the same shape as an outer surface of a corresponding part of distal portion 788.

Second skived portion 742B may be received within a port, such as port 506 or any of the other ports disclosed herein. Because second skived portion 742B has a non-round shape, second skived portion 742B may be keyed with respect to the port, to prevent torqueing/rotation between second skived portion 742B and the port. Protrusion 794 may interact with features (e.g., tension lock member 582A) to help to retain second skived portion 742B within the port.

First skived portion 742A and/or distal portion 788 may be received within a hub, such as any of the hubs disclosed herein. A non-round shape of first skived portion 742A may key first skived portion 742A with respect to an opening of the hub and may inhibit rotation between first skived portion 742A and the hub. Although not depicted in FIG. 11, shaft 740 may also include one or more wings (such as those discussed above) for securing shaft 740 to the hub.

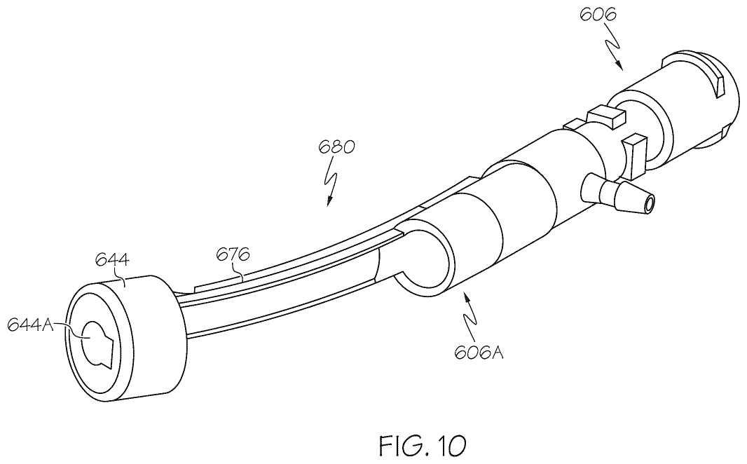

FIG. 12 shows an alternative shaft 840, which may be used along with aspects of the disclosure discussed above and below. In particular, shaft 840 may have any of the features of shaft 740, unless otherwise specified. A distal portion 888 of shaft 840 may have any of the features of distal portion 788. A skived portion 842 of alternative shaft 840 may have any of the features of first skived portion 742A.

Instead of second skived portion 742B, alternative shaft 840 may include a proximal portion 895, which may have the same properties as distal portion 888. Thus, only skived portion 842, between distal portion 888 and proximal portion 895 may be skived away. Proximal portion 895 may include all of the lumens of distal portion 888, including, for example, a working channel 808, fluid delivery lumens 814, a communication lumen 824, and wire lumens 826. A proximal face of proximal portion 895 may include a tapered portion 895A, which may be formed by removing a proximal wedge of proximal portion 895 near communication lumen 824. Tapered portion 895A may assist with securing proximal portion 895 to a port. Similarly to protrusion 794, discussed above, a distal face of proximal portion 895 may interact with features (e.g., tension lock member 582A) to help to retain proximal portion 895 within the port.

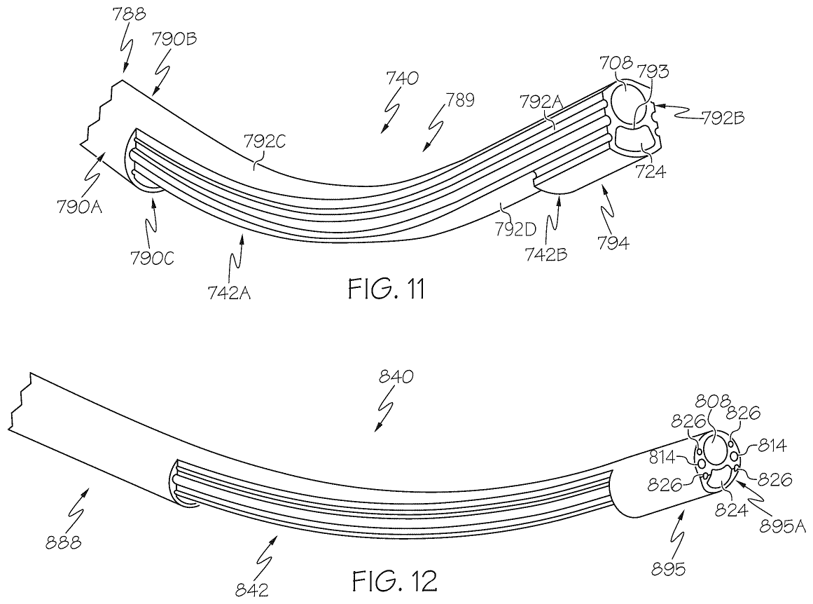

FIG. 13 depicts an alternative shaft 940, which may be used along with aspects of the disclosure discussed above and below. Shaft 940 may include a distal portion 988 and a skived portion 942. Skived portion 942 may extend from distal portion 988 to a proximalmost end of shaft 940. Alternatively, skived portion 942 may terminate proximally of the proximalmost end of shaft 940.

Proximal portion 988 may include protrusions 988A (e.g., tubular protrusions) extending from a central portion 988B. Central portion 988B may have an approximately tubular shape, and protrusions 988A may be diametrically opposed to one another, across central portion 988B. Protrusions 988A may be skived away from almost an entirety of shaft 940, including portions that are inserted into a subject (not shown). Protrusions 988A may only be retained at a portion of shaft 940 that is within or near to a hub (such as any of the hubs disclosed herein). Protrusions 988A may mate with corresponding features in a lumen of a hub in order to help secure proximal portion 988 to the hub. For example, protrusions 988A may inhibit shaft 940 from rotating relative to the hub.

Skived portion 942 may have portions 990A and 990B of proximal portion 988 skived away to form a first side surface 992A and a second side surface 992B. First side surface 992A and second side surface 992B may have any of the properties of first side surface 792A and second side surface 792B of FIG. 11, respectively. Skived portion 942 may also have a portion of proximal portion 988 that defines a communication lumen skived away. Skived portion 942 may be skived so that a portion of the communication lumen is skived away and a portion of the communication remains, leaving a recess 996 that corresponds to a radially inner surface of the communication lumen. Recess 996 may receive portions of frames, ports, or hubs to help to couple shaft 940 to the frame, port, or hub and to inhibit rotation of shaft 940 relative to the frame, port, or hub. The only full/complete lumen included in skived portion 942 may be a working channel 908.

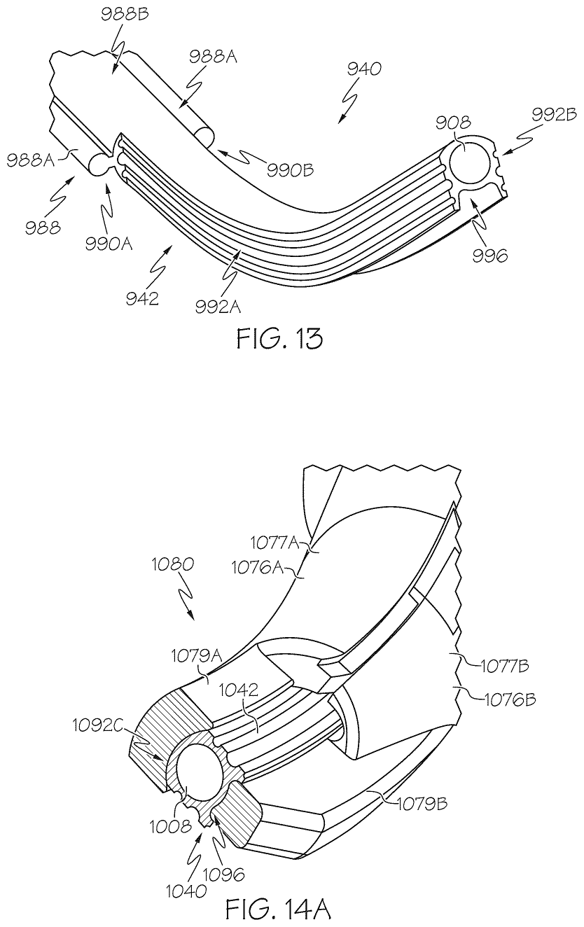

FIG. 14A depicts a portion of an exemplary frame 1080 that may have any of the features of any of the other frames disclosed herein. FIG. 14B depicts an exemplary handle 1002 including frame 1080. Frame 1080 may include a port 1006, having any of the features of any of the ports disclosed herein. Frame 1080 may also include a hub 1044, having any of the features of any of the hubs disclosed herein. Handle 1002 may also include a skived portion 1042 of a shaft 1040 which may include a working channel 1008. A body of handle 1002 is shown as being transparent in FIG. 14A to better depict aspects of frame 1080. Shaft 1040 may have any of the features of any of the shafts disclosed herein. Skived portion 1042 may have any of the features of any of the skived portions disclosed herein. For example, skived portion 1042 may have features of skived portion 942.

Frame 1080 may include a first beam 1076A extending from port 1006 to hub 1044. Frame 1080 may also include a second beam 1076B extending distally from port 1006 toward hub 1044 but terminating proximally of hub 1044. First beam 1076A and second beam 1076B may each include respective proximal portions 1077A and 1077B. Proximal portions 1077A and 1077B may surround an entirety of skived portion 1042 (e.g., radially surround skived portion 1042).

First beam 1076A may also include a distal portion 1079A, and second beam 1076B may include a distal portion 1079B. Distal portions 1079A and 1079B may surround only portions of skived portion 1042. For example, distal portion 1079B may be received within a groove or recess 1096 of skived portion 1042, having any of the features of recess 996. Distal portion 1079B may serve as a torque lock to inhibit skived portion 1042 from rotating relative to frame 1080. For example, ridges on either side of recess 1096 may retain distal portion 1079B within recess 1096. Distal portion 1079A may include a shape configured to mate with a side of skived portion 1042, opposite from recess 1096. For example, skived portion 1042 may include a surface 1092C having any of the features of third side 792C shown in FIG. 11. Distal portion 1079A may have a concave inner surface that complements a convex, curved shape of 1092C.

FIG. 15 depicts an exemplary shaft 1140, which may have any of the features of any of the other shafts disclosed herein. Shaft 1140 may include a skived portion 1142, having any of the features of any of the skived portions disclosed herein. Shaft 1140 may also include wings 1166, having any of the features of the wings disclosed herein.

Furthermore, exemplary shaft 1140 may include one or more notches 1172. Notches 1172 may be formed by punching out portions of shaft 1140 (e.g., of a communication lumen or any other lumen) or by other methods. One or more notches 1172 may mate with features of bodies of handles, hubs, and/or ports. For example, a notch 1172 may include a wall 1174, which may be keyed to structures in handle bodies, hubs, and/or ports, described above. Wall 1174 may have a curved distal portion and straight side portions. Notch 1172 may be open (e.g., not have a wall) on a proximal side of notch 1172. Notch 1172 may aid in securing shaft 1140 to handle bodies, hubs, and/or ports or in inhibiting shaft 1140 from rotating relative to the handle bodies, hubs, and/or ports.

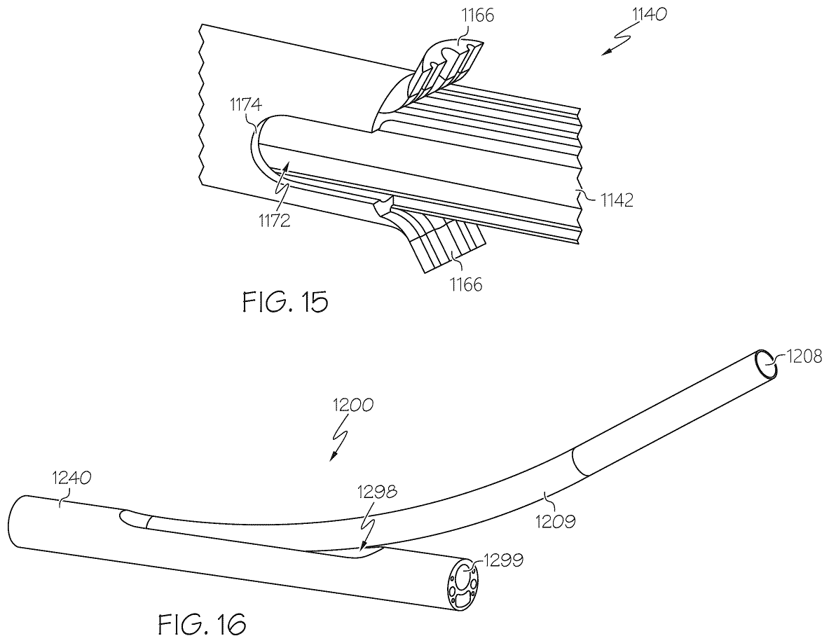

FIG. 16 depicts an alternative shaft assembly 1200. Shaft assembly 1200 may include a shaft 1240, having any of the features of any of the shafts disclosed herein. Instead of including a working channel defined by a multi-lumen extrudate, a separate tube 1209 defining a working channel 1208 may extend through at least a portion of shaft 1240. For example, tube 1209 may be received within a lumen 1299 of shaft 1240. Shaft 1240 may include an opening 1298 (e.g., an elongate opening or a slot) formed in a radially outer surface of shaft 1240. Opening 1298 may be in communication with lumen 1299 of shaft 1240. Opening 1298 may be formed by skiving shaft 1240. An entirety of opening 1298 may be distal to a proximalmost end of shaft 1240. Tube 1209 may extend through opening 1298 and into lumen 1299. A proximal end of tube 1209 may be coupled to a port, such as any of the ports disclosed herein. Adhesive may be added into opening 1298 to secure tube 1209 without concern about adhesive leaking into other lumens of shaft 1240.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device, comprising:
a handle, wherein the handle includes a port; and
a shaft extending from a portion of the handle, wherein a first portion the shaft includes a plurality of first lumens and a second lumen, wherein the plurality of first lumens and the second lumen are defined by a single, integrally formed body, wherein a second portion of the shaft defining the second lumen extends proximally of proximalmost openings of each of the plurality of first lumens, and wherein a proximal end of the second portion of the shaft is coupled to the port.

2. The medical device of claim 1, wherein the second lumen is a working channel.

3. The medical device of claim 1, wherein the plurality of first lumens includes at least one fluid delivery lumen.

4. The medical device of claim 3, wherein the plurality of first lumens further includes a plurality of wire lumens.

5. The medical device of claim 1, wherein the second portion of the shaft has a smaller width than the first portion of the shaft.

6. The medical device of claim 1, wherein the second lumen is an only lumen of the second portion of the shaft.

7. The medical device of claim 1, further comprising a coupling tube received within a lumen of the port and within the second lumen.

8. The medical device of claim 7, wherein a distal portion of the coupling tube includes an enlarged portion or a groove.

9. The medical device of claim 1, wherein the shaft includes a wing portion.

10. The medical device of claim 9, wherein the wing portion includes a first, attached distal end and a second, free end.

11. The medical device of claim 1, wherein a hub at least partially surrounds the shaft, and wherein the hub is coupled to the port by a beam.

12. The medical device of claim 11, wherein the beam is a first beam, and wherein a second beam extends distally from the port.

13. The medical device of claim 12, wherein the second portion of the shaft includes a recess, and wherein the first beam or the second beam is received within the recess.

14. The medical device of claim 1, wherein the second portion of the shaft is keyed to the port.

15. The medical device of claim 1, wherein a third portion of the shaft, proximal of the second portion of the shaft, includes a third lumen.

16. A medical device, comprising:
a handle, wherein the handle includes a port; and
a shaft extending from a portion of the handle, wherein the shaft includes a proximal portion and a distal portion, wherein the distal portion includes a plurality of lumens, wherein the proximal portion includes only a single lumen of the plurality of lumens, wherein the shaft is a single, integrally formed body, and wherein the proximal portion is coupled to the port.

17. The medical device of claim 16, further comprising a coupling tube received within a lumen of the port and within the single lumen of the proximal portion, and wherein a distal portion of the coupling tube includes an enlarged portion or a groove.

18. The medical device of claim 16, wherein a hub at least partially surrounds the shaft, and wherein the hub is coupled to the port by a beam.

19. A medical assembly, comprising:
a frame including a port, a hub, and a beam extending from the port to the hub; and
a shaft, wherein the shaft includes a proximal portion and a distal portion, wherein the shaft is a single, integrally formed body defining a plurality of lumens, wherein the proximal portion has a smaller width than the distal portion, wherein the proximal portion is coupled to the port, and wherein the hub is coupled to the proximal portion or the distal portion.

20. The medical assembly of claim 19, wherein the proximal portion includes a single lumen of the plurality of lumens, and wherein a remainder of the plurality of lumens have proximal ends that are distal to a proximal end of the single lumen.
